(19) United States
(12) Patent Application Publication
(10) Pub. No.: US 2024/0197251 A1
(43) Pub. Date: Jun. 20, 2024

(54) PATCH FOR ATTACHING A SENSOR TO HUMAN SKIN

(71) Applicant: Drägerwerk AG & Co. KGaA, Lübeck (DE)

(72) Inventor: Michael RIECKE, Lübeck (DE)

(21) Appl. No.: 18/541,126

(22) Filed: Dec. 15, 2023

(30) Foreign Application Priority Data
Dec. 19, 2022 (DE) 10 2022 133 935.1

(51) Int. Cl. A61B 5/00; A61B 5/01

(52) U.S. Cl. CPC A61B 5/6833; A61B 5/01; A61B 2560/0406

(57) ABSTRACT

A patch for attaching a sensor to human skin is provided, which includes an adhesive layer, a protective layer, and a sweat conduction element. The adhesive layer is arranged on an underside of the patch. The adhesive layer is configured to adhere to human skin. The protective layer is arranged on an upper side of the patch facing away from the underside. The patch has an area for receiving the sensor. The sweat conduction element runs in a base area of the patch. The sweat conduction element is connected to an external area of the patch in order to conduct sweat to the outside.

### CROSS REFERENCE TO RELATED APPLICATIONS

This application claims the benefit of priority under 35 U.S.C. § 119 of German Application 10 2022 133 935.1, filed Dec. 19, 2022, the entire contents of which are incorporated herein by reference.

### TECHNICAL FIELD

The present invention relates to a patch (plaster) for attaching a sensor to human skin.

### BACKGROUND

Patches for attaching a sensor to human skin are known.

With such patches, sweat can form in the area of the patch, especially when the wearer of the patch is under physical strain. This can cause the patch to peel off or impair the function of the sensor.

EP 3 849 402 B1 discloses a measuring device for measuring the body temperature of a living being, in particular a human being, with a measuring unit for measuring the body temperature. The measuring device has a first adhesive layer for attaching the measuring device to the body of the living being, the first adhesive layer being arranged on a side of the measuring device facing the body intended for this purpose. Furthermore, the measuring device has a skin-compatible protective layer which is arranged between the first adhesive layer and the measuring unit.

DE 10 2011 089 713 A1 discloses an electronic functional patch, the functional patch comprising a carrier structure, at least one electronic component which is arranged on the carrier structure, an adhesive layer and at least one absorbent body which is set up to absorb liquids. The absorbent body is arranged in the carrier structure and/or between the carrier structure and the adhesive layer.

Adhesive patches are also known in which ventilation openings are provided in a carrier layer of the patch in the form of perforations running in the height direction of the patch so that sweat can evaporate and be removed. However, such adhesive patches are not configured for attaching a sensor to human skin, as the perforations mean that the backing layer cannot act as a protective layer for a sensor, or can only act as such to a limited extent.

### SUMMARY

The present invention is therefore based on the task of providing a patch which is configured to attach a sensor to human skin and is insensitive to sweat.

These and other tasks are solved by the patch according to the invention.

According to the invention, a patch for attaching a sensor to human skin is provided, which comprises an adhesive layer, a protective layer and a sweat conduction element. The adhesive layer is arranged on an underside of the patch, wherein the adhesive layer is configured to adhere to human skin. The protective layer is arranged on an upper side of the patch facing away from the underside. The patch has an area for receiving the sensor. The sweat conduction element runs in a base area of the patch. The sweat conduction element is connected to an external area of the patch, namely to a side surface of the patch or to a peripheral surface of the patch, in order to conduct sweat to the outside.

In this way, a patch can be provided for attaching a sensor to human skin, which is set up by means of the sweat conduction element to absorb sweat formed in the area of the patch and drain it to the outside. In this way, it is possible to prevent sweat that forms over a period of use or examination time from accumulating under the patch and thus leading to the patch coming off or the functionality of the sensor being impaired.

As the sweat conduction element runs in a base area of the patch and is connected to the outer area of the patch, the sweat dissipation can be achieved without the protective layer having to be interrupted by perforations or other ventilation openings. In this way, the protective layer has a particularly good protective effect for the sensor.

A sensor is understood to be a device which is configured to generate a signal indicating at least one parameter, in particular a physical, chemical and/or biological parameter. The at least one parameter is particularly preferably a physiological parameter or a vital parameter, for example a heart rate, a respiratory rate, a blood pressure, a body temperature and/or a local heat flux density.

The sensor can have a measuring device or a transducer as a component to carry out the measurement. The sensor can have further components, which can be integrated together with the measuring device or the measuring sensor in a common housing on a common circuit board, for example a flexible circuit board. In particular, the sensor can have a control unit, such as a microprocessor, as a further component. The sensor can also have an energy storage device such as an accumulator or a battery as a further component in order to provide energy for the operation of the sensor. The sensor can have a data interface for data input and/or data output. The sensor can be set up for wireless data transmission via the data interface, for example. The sensor can be connected to a medical device, for example, via the data interface.

An adhesive layer is understood to be a layer of the patch arranged on an underside of the patch, which has an adhesive that is configured to adhere, i.e. stick, to human skin.

The adhesive layer can comprise only one adhesive layer with an adhesive or comprise a first adhesive layer with a first adhesive and a second adhesive layer with a second adhesive. The first adhesive layer and the second adhesive layer may be separated by a carrier layer (backing layer). The first adhesive and the second adhesive can be configured as similar or of different types of adhesives.

A protective layer is understood to be a layer of the patch arranged on the upper side of the patch, which is configured to protect the sensor from undesirable stresses. These can be mechanical and/or thermal stresses in particular.

It is particularly preferred that the protective layer of the patch is closed, i.e. that the protective layer has no openings such as perforations or ventilation openings. In this way, the protective layer has a particularly good protective effect.

In another particularly preferred embodiment, however, the protective layer of the patch has a recess in or on which the area for receiving the sensor can be formed. In this case, the adhesive layer forms a closed layer to act as a moisture barrier between the protective layer and the skin.

The patch can have other layers in addition to the adhesive layer and the protective layer.

An area for receiving (and/or mounting/holding) the sensor is understood to be an area within the patch or in or on an outer side of the patch, in particular on or in the underside of the patch, which is configured to receive the sensor.

The area for receiving the sensor can, for example, be formed as part of the adhesive layer and/or as part of the protective layer.

If the area for receiving the sensor is formed as part of the adhesive layer, the adhesive layer in the area for receiving the sensor can have different properties than the rest of the adhesive layer, for example a different adhesive can be provided there for receiving the sensor. Preferably, if the area for receiving the sensor is part of the adhesive layer, this part of the adhesive layer is formed in the same way as the rest of the adhesive layer and therefore has the same adhesive.

If the area for accommodating the sensor is formed as part of the protective layer, the sensor can be accommodated on or in the protective layer. The sensor can also be accommodated between the protective layer and the adhesive layer.

A sweat conduction element is an element that is configured to absorb sweat, conduct sweat towards the outer area of the patch and discharge sweat to the outside, i.e. release sweat.

A connection between the sweat conduction element and the outer area of the patch is understood to mean that the sweat conduction element is configured to provide a fluidic connection between the skin and the outer area of the patch with regard to sweat.

The sweat conduction element runs (at least partially, preferably completely) in a base surface of the patch.

A base area is a flat or curved area that is parallel to a patch surface area. In the case of a flat surface area, the base area therefore extends essentially perpendicular to a height direction of the patch.

As explained, the outer area of the patch is a side face of the patch or a circumferential area of the patch.

Preferably, the sweat conduction element is arranged in or on the adhesive layer or is formed as part of the adhesive layer. The sweat conduction element can extend partially or completely into the adhesive layer when viewed in the height direction, so that the adhesive layer can have a reduced thickness in the area of the sweat conduction element when partially extended or can be completely interrupted when fully extended.

In the event that the protective layer has a recess, it is preferred that the sweat conduction element does not extend completely into the adhesive layer when viewed in the vertical direction. In other words, in this case it is preferred that the adhesive layer is closed towards the protective layer and that the adhesive layer consequently acts as a moisture barrier towards the protective layer.

Preferably, the area for receiving the sensor does not have a sweat conduction element.

Preferably, the sweat conduction element comprises a sweat conduction material.

This makes it particularly easy to provide the sweat conduction element.

In this preferred embodiment, the sweat conduction element has a sweat-conducting effect in that it has a sweat-conducting material. The sweat conduction material can act as a kind of wick. A sweat-conducting material is a material that has a conductivity for sweat, in particular a capillary conductivity for sweat.

Preferably, the sweat conduction element is configured as a sweat conduction slit or slot.

In this way, the sweat conduction element can be provided with little material.

A slit is understood to be an elongated indentation at least in the adhesive layer.

In this preferred embodiment, the sweat conduction element has a sweat-conducting effect in that it is configured as a sweat-conducting slit (slot). It is particularly preferable for the slit to be configured as a capillary so that sweat is conducted along the capillary in the direction of the outer area due to surface effects.

Preferably, the patch has a plurality of sweat conduction elements.

In this way, the amount of sweat that can be discharged can be advantageously increased.

Preferably, each of the plurality of sweat conduction elements may comprise a sweat conductive material or each of the plurality of sweat conduction elements may be formed as a sweat conductive slit or a part of the plurality of sweat conduction elements may comprise a sweat conductive material and the other part of the plurality of sweat conduction elements may be formed as sweat conductive slits.

Preferably, the protective layer has a material for thermal insulation of the sensor.

In this way, the sensor can be protected from thermal influences that could falsify a measurement.

Preferably, the material for thermal insulation of the sensor is a foam material, particularly preferably a closed-cell foam such as a closed-cell PU foam.

Preferably, the adhesive layer has an adhesive selected from silicone adhesive, acrylic adhesive and/or hydrogel.

In this way, the adhesive behavior of the patch on human skin can be adapted to different applications. Acrylic adhesives can be suitable, for example, if a high, one-time adhesive strength is required. Hydrogel is advantageous for adhering to sensitive skin, such as the skin of a neonate. Silicone adhesive offers a good compromise between adhesive strength and usability on sensitive skin. Furthermore, silicone adhesives are often reusable.

Preferably, the sweat conduction material is a material selected from a porous plastic fiber composite, a sintered porous plastic and/or a textile.

As part of the development of the invention, it was recognized that these materials are particularly suitable for use as sweat conduction materials.

The preferred textile is a yarn, a nonwoven fabric and/or a felt. Preferably, the textile is produced on the basis of cotton and/or cellulose. For example, the textile can be provided as a cotton yarn or as a cellulose yarn.

Preferably, the patch has an electrically conductive element in or on the adhesive layer, the electrically conductive element being adapted to form an electrical connection between the human skin and the sensor.

In this way, an electrical connection can be established between the skin and the sensor in order to measure electrical parameters. An example of such an electrical parameter is the electrical activity of the heart, which indicates the heart rate.

The patch can have a number of electrically conductive elements.

The electrically conductive element or the plurality of electrically conductive elements can be provided in essentially any configuration. However, it is particularly advantageous to provide them in the form of electrically conductive films, wires and/or fibers (for example as carbon fibers), which can be introduced into or applied to the adhesive layer.

Preferably, the protective layer is configured to form a first structural unit, wherein the adhesive layer is configured to form a second structural unit and wherein the second structural unit can be detachably connected to the first structural unit, so that the first structural unit can be reused.

In this variant of the invention, the adhesive layer is arranged on the underside of the patch when the first structural unit and the second structural unit are connected.

In this way, the patch can be advantageously provided as a reusable patch, in particular in that the first structural unit is suitable for multiple uses.

In order to bring the patch into a state of use, the second structural unit can be connected to the first structural unit by a user and applied to the skin of the user or another person. After use, the patch can be removed, and the second structural unit detached from the first structural unit and reprocessed or disposed of. The first structural unit can be reused and connected to a further or reprocessed second structural unit as described above.

The first structural unit can have the sensor as an integral component. In this way, the manageability of the patch can be improved, as the first unit does not have to be assembled by a user.

Alternatively, the sensor can be provided separately and can be connected to the protective layer to form the first structural unit. In this way, the patch can be adaptable to different conditions of use in that a user only has to select the sensor suitable for the respective condition of use and assemble the selected sensor with the protective layer to form the first unit.

Both the first unit and the second unit can have additional elements.

Particularly preferably, the first unit has the sensor and an external cover layer, with the area for accommodating the sensor being arranged between the protective layer and the cover layer.

Thus, the second structural unit is detachably connectable to the cover layer in order to be detachably connectable to the first structural unit. In other words, in this particularly preferred embodiment, the connection between the first unit and the second unit is made by bonding the adhesive layer to the cover layer. Stress on the sensor can be reduced by the cover layer. The patch can thus be provided in a particularly advantageous manner for reuse, as the sensor is protected by a cover layer.

Preferably, the cover layer, sensor and protective layer are integrally connected to each other and thus form the first structural unit of the patch. The cover layer and protective layer, and preferably also the cover layer and sensor, can be connected by means of an adhesive. An adhesive, for example, is a suitable bonding agent.

It is preferred that the adhesive layer comprises a first adhesive layer with a first adhesive and a second adhesive layer with a second adhesive, wherein the first adhesive layer and the second adhesive layer are separated by a carrier layer. In this way, the removability of the adhesive layer from the first structural unit or from the cover layer, if present, is improved. It is preferred that the first adhesive and the second adhesive are configured as different adhesives. Particularly preferred is that the first adhesive, which is configured for attachment to the skin, is a silicone adhesive and the second adhesive, which is configured for attachment to the first structural unit or to the cover layer, if present, is a comparatively stronger adhesive, such as an acrylic adhesive or a silicone adhesive. In this way, the detachability of the adhesive layer from the first structural unit or from the cover layer, if present, is improved in that the patch can be detached as a whole from the skin in a first step and the adhesive layer can be detached from the first structural unit or from the cover layer in a second step.

Preferably, the sensor is configured as a temperature sensor and/or as a heat flow sensor.

The patch according to the invention can thus be used particularly advantageously for measuring thermal parameters such as body temperature.

If the sensor is configured as a heat flow sensor, it is preferable that the area for receiving the sensor is located between the adhesive layer and the protective layer.

It is also preferred that the protective layer has the aforementioned recess, so that a heat flow from the skin through the heat flow sensor to an environment is influenced as little as possible by the protective layer and the measuring behavior can thus be advantageously improved.

In a preferred variant of the invention, the patch has the sensor. Particularly preferably, the patch has the sensor as an integral component.

These and other features of the invention are also apparent from the following description of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a sectional view of an embodiment of a patch according to the invention;

FIG. 2 is a sectional view of an embodiment of a patch according to the invention;

FIG. 3 is a sectional view of an embodiment of a patch according to the invention;

FIG. 4 is a bottom view showing an embodiment of a patch according to the invention;

FIG. 5 is a bottom view showing an embodiment of a patch according to the invention;

FIG. 6 is a bottom view of an embodiment of a patch according to the invention;

FIG. 7 is a bottom view of an example of a patch according to the invention;

FIG. 8 is a sectional view of an embodiment of a patch according to the invention;

FIG. 9 is a sectional view of an embodiment of a patch according to the invention;

FIG. 10a is a sectional view of an embodiment of an adhesive layer according to the invention; and

FIG. 10b is a sectional view of an embodiment of an adhesive layer according to the invention.

### DESCRIPTION OF PREFERRED EMBODIMENTS

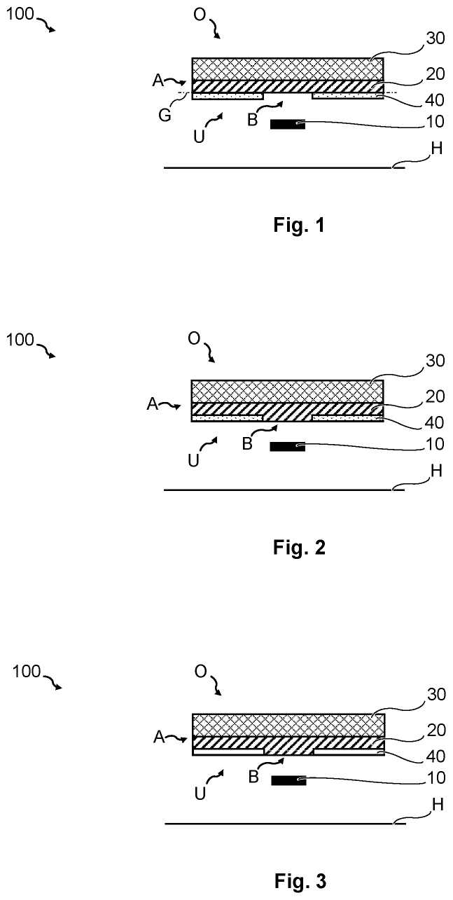

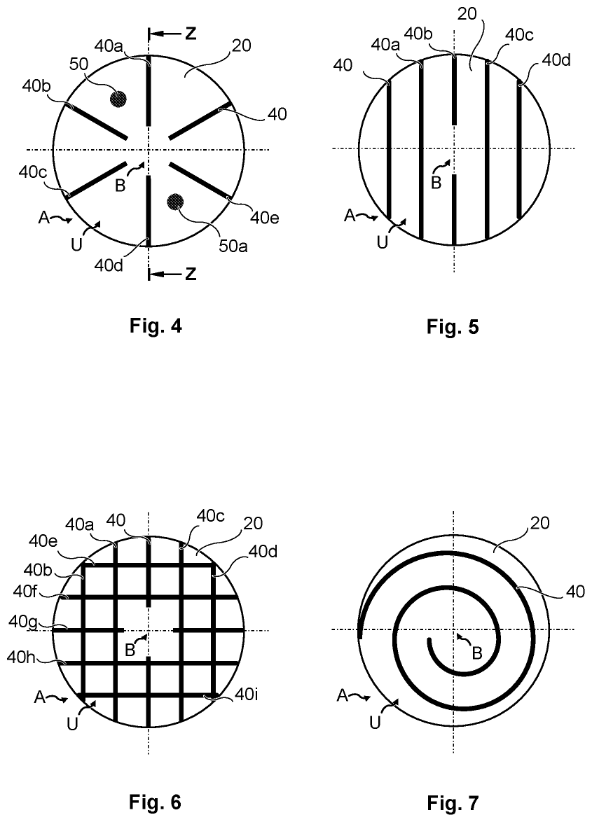

Referring to the drawings, as shown in FIGS. 1-9, a patch 100 for attaching a sensor 10 to human skin H is provided according to the invention. FIGS. 4-7 show lower views of patches 100 according to the invention. FIGS. 1-3 and 8-9 show sectional views of patches 100 according to the invention. For example, the sectional views may be sections along a sectional axis Z-Z, as shown as an example in FIG. 4.

FIGS. 1-3 show schematically that the sensor 10 can be provided separately from the patch 100.

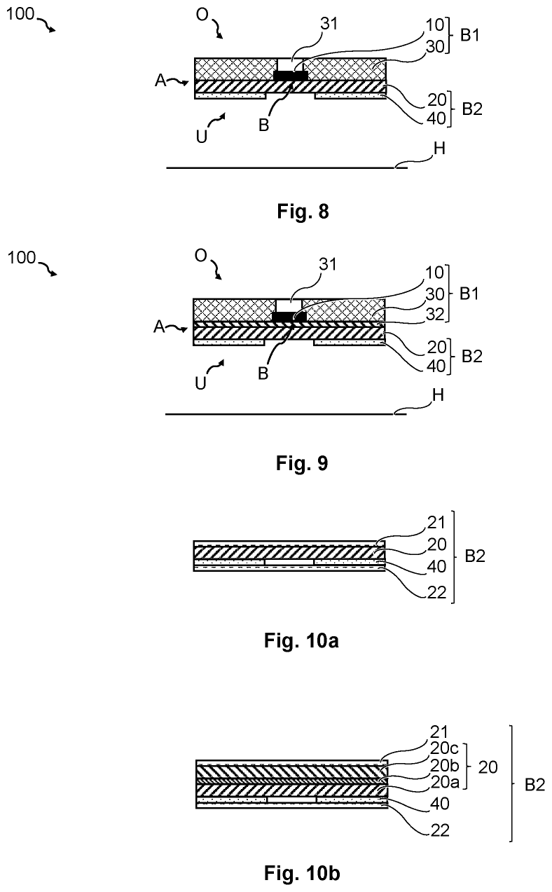

However, the sensor 10 can also be provided as an integral part of the patch 100, as shown schematically in FIGS. 8-9.

The patch 100 may be substantially planar (flat), as shown in FIGS. 1-3 and 8-9, or, in an embodiment not shown, may be non-planar, for example multi-dimensionally curved, for example to secure the patch 100 to a sternum.

As shown in FIGS. 4-7, the patch 100 can have an essentially circular basic shape or, in an embodiment not shown, a basic shape deviating from this basic shape.

The patch 100 has an adhesive layer 20, a protective layer 30 and a sweat conduction element 40.

The adhesive layer 20 is arranged on an underside U of the patch 100 in an assembled state of the patch 100, wherein the adhesive layer 20 is configured to adhere to human skin H.

The protective layer 30 is arranged on an upper side O of the patch 100 facing away from the lower side U, whereby the patch has an area B for receiving (mounting) the sensor 10.

The area B for receiving the sensor 10 can, for example, be arranged in a central area of the underside U, as shown in FIGS. 4-7. The sensor 10 is accommodated between the skin H and the adhesive layer 20.

The area B for receiving the sensor 10 can alternatively be formed as part of the adhesive layer 20 and/or as part of the protective layer 30, as shown in FIGS. 8 and 9. In this case, the sensor 10 is accommodated within the patch 100.

The sweat conduction element 40 runs in a base surface G of the patch 100, as indicated in FIG. 1. In the embodiment example shown, this is achieved by the sweat conduction element 40 bordering on the base surface G.

The sweat conduction element 40 is connected to an external area A, namely to a side surface of the patch 100 or to a peripheral surface of the patch 100, in order to dissipate (drain, move by capillary action)) sweat to the outside.

The sweat conduction element 40 can be arranged on the adhesive layer, as shown in the embodiment examples according to FIGS. 1, 8 and 9.

The sweat conduction element 40 can be arranged in the adhesive layer 20, as shown in the embodiment examples according to FIGS. 2 and 3.

It is particularly preferred and shown in FIGS. 1 to 3 that the protective layer 30 is closed, i.e. has no openings such as perforations or other ventilation openings.

In another preferred variant of the invention, which is shown in FIGS. 8 and 9, the protective layer can have a recess 31. Preferably, the area B for receiving the sensor 10 is formed in or on the recess.

It is preferred and shown in FIGS. 4 to 7 that the area B for receiving the sensor 10 does not have a sweat conduction element 40.

It is preferred and shown in FIGS. 1 and 2 that the sweat conduction element 40 can comprise a sweat conduction material.

It is preferred and shown in FIG. 3 that the sweat conduction element 40 can be configured as a sweat conduction slit (or slot).

As shown in FIG. 3, the slit can only partially extend into the adhesive layer 20 when viewed in the vertical direction.

In an alternative not shown, the slit can extend completely through the adhesive layer 20 when viewed in the vertical direction, i.e. up to the protective layer 30 or even into the protective layer 30.

It is preferred and shown in FIGS. 4 to 6 that the patch 100 can have a plurality of sweat conduction elements 40, 40a, 40b, . . . 40i.

Each of the plurality of sweat conduction elements 40, 40a, 40b, . . . 40i can have a sweat conduction material or each of the plurality of sweat conduction elements 40, 40a, 40b, . . . 40i can be configured as a sweat conduction slit or a part of the plurality of sweat conduction elements 40, 40a, 40b, . . . 40i can have a sweat conduction material and the other part of the plurality of sweat conduction elements 40, 40a, 40b, . . . 40i can be configured as sweat conduction slits.

In this respect, FIG. 4 shows an embodiment example of a patch 100 in which the plurality of sweat conduction elements 40, 40a, 40b, . . . 40e extend in a star shape from the area B for receiving the sensor 10 in the direction of the outer area A. Preferably, each of the plurality of sweat conduction elements 40, 40a, 40b, . . . 40e is formed as a sweat conduction material or as a sweat conduction slit.

FIG. 5 shows an embodiment example of a patch 100 in which the plurality of sweat conduction elements 40, 40a, 40b, . . . 40d extends from the area B for receiving the sensor 10 in the direction of the outer area A, the sweat conduction elements 40, 40a, 40b, . . . 40d being parallel to one another. Preferably, each of the plurality of sweat conduction elements 40, 40a, 40b, . . . 40e has a sweat conduction material.

FIG. 6 shows an embodiment example of a patch 100 in which the plurality of sweat conduction elements 40, 40a, 40b, . . . 40i is divided into two groups. The sweat conduction elements 40, 40a, 40b, . . . 40i of the first group are parallel to each other and the sweat conduction elements 40, 40a, 40b, . . . 40i of the second group are also parallel to each other. The plurality of sweat conduction elements 40, 40a, 40b, . . . 40i extend from the area B for receiving the sensor 10 in the direction of the outer area A. The sweat conduction elements 40, 40a, 40b, . . . 40i of the first groups form a predetermined angle with the sweat conduction elements 40, 40a, 40b, . . . 40i of the second groups, in the case shown a predetermined angle of 90 degrees, so that the plurality of sweat conduction elements 40, 40a, 40b, . . . 40i together form a kind of grid arrangement. Preferably, each of the plurality of sweat conduction elements 40, 40a, 40b, . . . 40e has a sweat conduction material.

FIG. 7 shows an embodiment example of a patch 100 which only has a sweat conduction element 40 which, as shown, extends in a spiral from the area B for receiving the sensor 10 in the direction of the outer area A. Preferably, the sweat conduction element 40 includes a sweat conduction material.

Preferably, the protective layer 30 in the illustrated embodiments includes a material for thermal insulation of the sensor 10. Preferably, the material for thermal insulation of the sensor 10 is a foam material, particularly preferably a closed-cell foam such as a closed-cell PU foam.

Preferably, the adhesive layer 20 in the illustrated embodiments includes an adhesive selected from silicone adhesive, acrylic adhesive and hydrogel.

Preferably, the sweat conduction material in the illustrated embodiments comprises a material selected from a porous plastic fiber composite, a sintered porous plastic and a textile.

Preferably, and as shown in FIG. 4, the patch 100 may comprise an electrically conductive element 50 in or on the adhesive layer 20, wherein the electrically conductive element 50 is configured to form an electrical connection between the human skin H and the sensor 10.

It is also shown in FIG. 4 that the patch 100 can have a plurality of electrically conductive elements 50, 50a. In the example shown, the plurality of electrically conductive elements 50, 50a are formed as electrically conductive films. It is not visible that the patch 100 can have conductors running between the electrically conductive elements 50, 50a and the area B for receiving the sensor 10 within the patch 100, so that when the sensor 10 is received in the area B, the electrical connection with the electrically conductive elements 50, 50a can be achieved via the conductors.

When providing a plurality of electrically conductive elements 50, 50a, it is preferable that these are located on the outside in a transverse direction of the patch 100, since in this way the receptivity of signals can be improved.

In all embodiments of the patch 100, the protective layer 30 may be configured to form a first structural unit B1, wherein the adhesive layer 20 may be configured to form a second structural unit B2 and wherein the second structural unit B2 is detachably connectable to the first structural unit B1, so that the first structural unit B1 is reusable. This is shown by way of example in the embodiment example according to FIG. 8 and in the embodiment example according to FIG. 9.

In this embodiment of the invention, the adhesive layer 20 is thus arranged on the underside U of the patch 100 in the connected state of the first structural unit B1 and the second structural unit B2.

In the embodiment example according to FIG. 8, the sensor 10 can be configured as an integral component of the first component B1 or can be provided as a separate component of the patch 100.

Both the first structural unit B1 and the second structural unit B2 can have further elements.

It is particularly preferred and shown in FIG. 9 that the first structural unit B1 can also have an outer cover layer 32 (in relation to the first structural unit B1), wherein the region B for receiving the sensor 10 is arranged between the protective layer 30 and the cover layer 32. The cover layer 32 can, for example, be configured as a plastic film, such as a polyethylene film.

In the embodiment example according to FIG. 9, the second structural unit B2 is detachably connectable to the cover layer 32 so as to be detachably connectable to the first structural unit B1.

In the embodiment shown in FIG. 9, the cover layer 32, sensor 10 and protective layer 30 are integrally connected to each other and thus form the first structural unit B1 of the patch.

Cover layer 32 and protective layer 30 and preferably also cover layer 32 and sensor 10 can be connected, for example, by means of an adhesive. An adhesive, for example, is suitable as a bonding agent.

FIGS. 10a and 10b show two variants of a second unit B2.

In both variants, the second structural unit B2 comprises the adhesive layer 20 and the sweat conduction element 40 or the plurality of sweat conduction elements 40.

In a state prior to a connection with the first structural unit B1, the second structural unit B2 may further comprise separating layers 21, 22 on external sides, which function to protect the second structural unit B2. The upper separating layer 21 can be removed by a user before the second structural unit B2 is connected to the first structural unit B1, thus exposing the adhesive layer 20. The lower separating layer 22 may be removed by a user prior to connection to the skin H, thereby exposing the adhesive layer 20 and the sweat conduction element 40 or the plurality of sweat conduction elements 40.

In the variant shown in FIG. 10a, the adhesive layer 20 only has one layer with an adhesive.

In the variant shown in FIG. 10b, the adhesive layer 20 has several layers 20a, 20b and 20c. In this respect, the adhesive layer 20 comprises a first adhesive layer 20a with a first adhesive and a second adhesive layer 20c with a second adhesive. The first adhesive layer 20a and the second adhesive layer 20c are also separated by a carrier layer 20b. The first adhesive and the second adhesive can be of the same or different types.

In all the embodiments of the patch 100 shown, the sensor 10 can be configured as a temperature sensor and/or as a heat flow sensor.

If the sensor 10 is configured as a heat flow sensor, it is preferable that the area for receiving the sensor 10 is arranged between the adhesive layer 20 and the protective layer 30, as shown in FIGS. 8 and 9.

In this case, it may be advantageous for the protective layer to have a recess 31, as also shown in FIGS. 8 and 9. Such a recess 31 minimizes the heat flow from the skin H through the heat flow sensor to an environment through the protective layer 30, which can be advantageous for certain configurations and sensors.

In all embodiments of the patch 100, the patch 100 may comprise the sensor 10.

While specific embodiments of the invention have been shown and described in detail to illustrate the application of the principles of the invention, it will be understood that the invention may be embodied otherwise without departing from such principles.

### LIST OF REFERENCE SYMBOLS

10 Sensor
20 Adhesive layer
20 First adhesive layer
20b Carrier layer
20c Second adhesive layer
21, 22 Separating layer
30 Protective layer (coating)
31 Recess
32 Cover layer
40, 40a, 40b, . . . ,40i Sweat conduction element
50, 50 Electrically conductive element
100 Patch
A External area
B Area for receiving the sensor
B1 First structural unit
B2 Second structural unit
G Base area (area)
H Human skin
O Top side
U Underside
Z-Z Section line

What is claimed is:

1. A patch for attaching a sensor to human skin, the patch comprising:
an adhesive layer arranged on an underside of the patch, the adhesive layer being adapted to adhere to human skin;
a protective layer arranged on an upper side of the patch, facing away from the underside; and
a sweat conduction element,
wherein the patch has an area for receiving the sensor,
wherein the sweat conduction element extends in a base surface of the patch, and
wherein the sweat conduction element is connected to a side surface of the patch or to a peripheral surface of the patch to dissipate sweat to a patch outside.

2. A patch according to claim 1, wherein the sweat conduction element comprises a sweat conduction material.

3. A patch according to claim 1, wherein the sweat conduction element is configured as a sweat conduction slit.

4. A patch according to claim 1, wherein the patch comprises a plurality of sweat conduction elements including the sweat conduction element and at least another sweat conduction element.

5. A patch according to claim 1,
wherein the protective layer comprises a material for thermal insulation of the sensor, and/or
wherein the adhesive layer comprises an adhesive selected from silicone adhesive, acrylic adhesive and/or hydrogel.

6. A patch according to claim 2, wherein the sweat conduction material comprises a material selected from a porous plastic fiber composite, a sintered porous plastic and/or a textile.

7. A patch according to claim 1, further comprising an electrically conductive element in or on the adhesive layer, wherein the electrically conductive element is arranged to form an electrical connection between the human skin and the sensor.

8. A patch according to claim 1,
wherein the protective layer is configured to form a first structural unit,
wherein the adhesive layer is arranged to form a second structural unit, and
wherein the second structural unit is detachably connected to the first structural unit, so that the first structural unit is reusable.

9. A patch according to claim 1, wherein the sensor is configured as a temperature sensor and/or as a heat flow sensor.

10. A patch according to claim 1, further comprising the sensor.

11. A patch according to claim 1, wherein the protective layer is not interrupted by perforations or other ventilation openings.
